(19) United States
(12) Patent Application Publication
(10) Pub. No.: US 2004/0213838 A1
(43) Pub. Date: Oct. 28, 2004

(54) Medical food tablets containing free amino acids

(76) Inventors: Terrence B. Mazer, Reynoldsburg, OH (US); Amy Marchio, Centerburg, OH (US); Phyllis Acosta, Westerville, OH (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 10/422,296

(22) Filed: Apr. 24, 2003

(51) Int. Cl. A61K 9/20; A61K 31/198

(52) U.S. Cl. 424/464; 514/561; 514/419; 514/423; 514/565; 514/400; 514/567

(57) ABSTRACT

Disclosed are medical foods tablets comprising (a) free amino acids, (b) vitamins, minerals, or combinations thereof, and (d) sweetening agents, wherein the tablets are preferably small and chewable, and are preferably prepared by dry mixing and then compressing the various ingredients into tablet form. The tablets are preferably substantially free of saturated fats and reducing sugars. Also disclosed are dosage form kits and opaque packages, comprising the tablets. All are useful in providing medical foods to, and improved dietary compliance among, individuals with metabolic disorders such as phenylketonuria or other afflictions responsive to selected amino acid diets. The small tablets have improved taste and/or aroma.

### TECHNICAL FIELD

The present invention relates to oral medical foods in tablet form comprising selected amino acids and other nutrients, all of which are useful for individuals afflicted with various inherited metabolic disorders responsive to restricted amino acid diets.

### BACKGROUND OF THE INVENTION

There are a variety of inherited metabolic disorders for which amino acids resulting from the digestion of proteins in a normal diet are incompatible. To effectively deal with each disorder, the afflicted individual must eat a special diet that includes selected amino acid blends. These selected blends are typically in the form of certain medical foods in combination with other nutrients compatible with the particular metabolic disorder.

Medical foods for use in managing various metabolic disorders include medical food products in the form of tablets, capsules, powders, or other product forms, each of which contains the particular blend of amino acids or other nutrients best suited for treatment of the particular metabolic disorder at issue. Examples of metabolic disorders that require or can benefit from such medical foods include phenylketonuria, branched chain keto aciduria, tyrosinemia, isovaleric acidemia, homocystinuria, urea cycle disorders, propionic acidemia, methylmalonic acidemia, and glutaric aciduria type 1.

Individuals taking these medical foods often find the taste and odor unpleasant, and in many instances the disagreeable nature of the products results in poor compliance in adults as well as children. In most instances, it is the blend of free amino acids that contributes most of the unpleasant flavor or odor. The offensive tasting products often come in the form of powders or other product forms that are mixed with water or other fluid prior to administration. These powders and drinks are messy and inconvenient to use throughout the day, especially for children at school and adults at work, and often have a highly disagreeable odor and taste. Much of the research over the years has therefore been directed to formulating medical foods that have better taste and aroma, are more convenient to use than powders, and will therefore result in improved patient compliance.

One common method of improving these medical foods involves the formulation of tablets rather than powders. Although these tablets are more convenient and less messy to use than powders, they often have their own unpleasant taste and aroma that can significantly reduce compliance. Moreover, these tablets are often large and difficult to swallow, and if formulated into smaller size dosage forms, results in the administration of an unrealistically large number of foul tasting or smelling tablets every day.

Still other methods to improve the odor and taste in these medical foods have often been directed to the formulation of sealed capsules or similar other product forms. Individuals swallow these capsules whole, and thus experience little or none of the inherently unpleasant taste and aroma from the amino acid blends sealed within the capsule. These sealed product forms, however, are often large and difficult to swallow, especially for young children, so that many individuals over time become frustrated and non-compliant.

Still other methods of improving upon the flavor and odor of these medical foods has involved the separation of the bad smelling amino acids from the other nutrients, so that the non-offensive nutrients are taken in one dosage form while the offensive nutrients are sequestered and delivered in a smaller, more acceptable, dosage form. This approach, however, typically results in the individuals taking even more tablets or capsules throughout the day.

It is therefore an object of the present invention to provide medical foods having improved taste and/or aroma. It is a further object of the present invention to also provide a method of improving compliance among individuals who could benefit from such medical foods, and further to provide such medical foods for improved ease of use and compliance, especially among children at school and adults at work.

### SUMMARY OF THE INVENTION

The present invention is directed to medical foods in tablet form comprising (a) a dry mix blend of free amino acids, (c) vitamins, minerals, or combinations thereof, and (d) sweetening agents, wherein the tablets are prepared by dry mixing the ingredients and compressing the dry mixed ingredients into tablet form. The free amino acids are preferably dry compacted into flowable particulates for use in and prior to the dry mixing step with the other ingredients.

The present invention is also directed to medical foods in the form of chewable tablets comprising (a) a blend of free amino acids, (b) vitamins, minerals, or combinations thereof, and (c) sweetening agents, wherein the tablets are chewable and have a major axis of less than about 1.5 cm and a ratio of a major to minor axis from about 1 to about 10.

The present invention is also directed to medical foods in the form of oral tablets comprising (a) a blend of free amino acids, (b) vitamins, minerals, or combinations thereof, (c) sweetening agents containing less than 10% by weight of a reducing sugar, (d) less than about 10% by weight of fat, wherein components (a), (b) and (c) represent at least about 70%, preferably from about 90% to 100%, by weight of the medical food tablet.

The present invention is also directed to packaged compositions comprising the medical food tablets described herein, wherein the packaged composition comprises a light resistant or opaque container and contained therein a plurality of medical food tablets of the present invention. It has been found that the medical food tablets as described herein discolor over time, and that such discoloration can be minimized or avoided by the use of a light resistant or opaque package or cover.

The present invention is also directed to a dosage form kit comprising (a) a first package having a resealable opaque container and a plurality of oral medical food tablets contained therein, and (b) a resealable second package removably positioned in the dosage form kit, said second package used to transport therein at least a portion of the tablets away from the dosage form kit, wherein the transported tablets are orally administered within days or weeks after transport. The dosage form kit is especially useful for children and adults to transport limited quantities of the tablets to school and work, respectively, or other daily or weekly activity.

The present invention is also directed to a method of providing nutrition for individuals afflicted with metabolic disorders such as phenylketonuria, wherein the method comprises oral administration of the medical food tablet embodiments substantially free of phenylalanine as described herein.

It has been found that the medical food tablets of the present invention have improved taste and aroma, and are especially useful when formulated and administered as small, chewable tablets, especially among young school age children who do not easily swallow capsules or otherwise take or chew large tablets.

### DETAILED DESCRIPTION OF THE INVENTION

The medical food tablets and corresponding methods and packaged embodiments of the present invention comprise limitations directed to free amino acid blends and other nutrients and preferred dry mixing process features. These and other essential elements or limitations of the present invention are described in detail hereinafter.

The term “medical food tablet” as used herein, unless otherwise specified, refers to medical foods in the form of solid, tabletted dosage forms suitable for administration by mouth, either by swallowing whole or preferably by chewing, and which help provide a selected amino acid diet to individuals afflicted with metabolic disorders responsive to such selected amino acid diets. The medical food tablets thus specifically exclude dosage forms such as powders that are typically intended for dilution prior to administration and gelatin and other capsules which are typically intended to be swallowed whole during administration.

The term “oral tablet” as used herein, unless otherwise specified, means chewable or non-chewable tablets to be administered by mouth. Chewable tablets are preferred.

The term “lipid” as used herein, unless otherwise specified, means fats, oils, and combinations thereof.

The term “children” as used herein, unless otherwise specified, means individuals between the ages of 4 and 18 years, typically from about 6 years to about 14 years, more typically from about 8 years to about 14 years.

All percentages, parts and ratios as used herein are by weight of the total composition, unless otherwise specified. All such weights as they pertain to listed ingredients are based on the active level and, therefore, do not include solvents or by-products that may be included in commercially available materials, unless otherwise specified.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, 5, 6, from 1 to 9, from 3.6 to 4.6, from 3.5 to 9.9, and so forth.

All references to singular characteristics or limitations of the present invention shall include the corresponding plural characteristic or limitation, and vice versa, unless otherwise specified or clearly implied to the contrary by the context in which the reference is made.

All combinations of method or process steps as used herein can be performed in any order, unless otherwise specified or clearly implied to the contrary by the context in which the referenced combination is made.

The compositions of the present invention can comprise, consist of, or consist essentially of the essential elements and limitations of the invention described herein, as well as any additional or optional ingredients, components, or limits disclosed herein or otherwise suitable for use in medical food tablets.

### Product Form

The medical food tablets of the present invention are oral product forms, preferably chewable tablets, comprising the amino acids and nutrients as described herein. These tablets are preferably formed by dry mixing methods as also described herein, and packaged in accordance with the preferred packaging and dosage form kit embodiments of the present invention.

The medical food tablets of the present invention are preferably small tablets that can easily be transported and periodically consumed by the intended user throughout the day. These small tablets have a preferred major axis of not more than about 1.5 cm, more preferably not more than about 1.2 cm, even more preferably not more than about 1 cm, wherein the tablets also have a preferred ratio of a major to minor axis of at least about 1, more preferably from about 1 to about 10, even more preferably from about 2 to about 6, and even preferably from about 2 to about 5. These tablets preferably have an individual total weight per tablet of less than about 1.5 gm, more preferably from about 0.5 gm to about 1.3 gm, most typically from about 0.9 gm to about 1.1 gm.

To help maintain the small, convenient size or configuration of the oral tablets, it is preferred that the combination of (a) free amino acids, (b) vitamins, minerals, or combinations thereof, and (c) sweetening agents, represent at least about 70%, preferably from about 75% to 100%, even more preferably from about 90% to 99%, by weight of the oral tablets.

The preferred major axis of the oral tablets of the present invention is defined herein as the longest cross-sectional distance, typically a symmetrical cross section, of the tablet, whereas the minor axis is the smallest symmetrical cross-sectional distance of the tablet. The tablets can take on any shape or configuration, whether symmetrical or asymmetrical, scored or not scored, with or without engraved or embossed designs or symbols, including oval, capsule form, spherical, cuboidal, conventionally round and raised, or any other known or otherwise suitable tablet shape or configuration, provided that the preferred embodiments as described above also have the requisite size configurations.

The oral tablets are preferably contained or dispensed within a light resistant or opaque package, cover or film coating. It has been found that the medical food tablets of the present invention can rapidly discolor over time, and that protection of the tablets from light during prolonged storage helps minimize or eliminate the development of the discolored appearance. Light protection is preferably in the form of a light resistant or opaque package, which can be in the form of single or unit dose packaging, multi-dose packaging, or combinations thereof.

The oral tablets of the present invention are preferably contained within a dosage form kit that provides a convenient packaging system for transporting tablets on a daily, weekly, or monthly basis away from home, e.g. to school, to the office or workplace, etc. The dosage form kit is especially useful for school age children in need of a convenient way to transport their prescribed medical food to school every day. The kit comprises (a) a first package having a resealable opaque or light resistant container and a plurality of oral tablets contained therein, and (b) a resealable second package removably positioned in or to the dosage form kit, said second package used to transport therein at least a portion of the oral tablets away from the dosage form kit, wherein the transported tablets are orally administered within about one month after transport, preferably within about one week after transport, more preferably within about 8-48 hours after transport, most typically within about 24 hours of transport.

The first package component of the dosage form kit can be any known or otherwise suitable resealable package for containing a plurality of solid oral tablets, non-limiting examples of which include conventional prescription vials, flexible pouches or bags with a resealable opening, and so forth. The resealable second package can likewise be any known or otherwise suitable package for containing the desired number of transported solid oral tablets, and is preferably a flexible pouch or bag with a resealable opening.

The term “resealable” as used herein, unless otherwise specified, means that the package or container to which it refers can be repeatedly closed and opened by the user so that the tablets therein cannot readily fall out when the package or container is sealed, but can still be easily removed if desired when the resealable container is opened again. The “resealable” aspect of these packages and containers preferably limits the exchange of air and/or moisture while the package or container is sealed, and provides little or no such limit when the package or container is open and yet to be resealed.

### Nutrients

The oral tablets of the present invention are medical foods that comprise sufficient types and amounts of nutrients to help meet the prescribed nutrient needs of the intended user. These tablets may therefore comprise various combinations of lipids, proteins and other amino nitrogen sources, carbohydrates, vitamins and minerals, and other materials, provided that each combination is safe and effective in helping meet the special nutrient needs created by the metabolic disorder of the individual for whom the tablets are intended.

All of the medical food tablets of the present invention comprise free amino acids, alone or preferably in combination with other nutrients. These free amino acids as well as other optional nutrients for use in the medical food tablets are described in greater detail hereinafter.

1. Free Amino Acids

The medical food tablets of the present invention comprise a blend of free amino acids, preferably dry blended amino acids, wherein the blend is defined by the nutrient needs created by the particular metabolic disorder of the individuals for whom the tablets are intended. The amino acids preferably provide or help provide each tablet with a protein equivalent of from about 0.2 gm to about 1.0 gm, more preferably from about 0.3 gm to about 0.8 gm, more preferably from about 0.3 gm to about 0.6 gm.

The free amino acids suitable for use in the medical food tablets of the present invention include L-alanine, L-arginine, L-asparagine, L-aspartic acid, L-carnitine, L-cystine, L-glutamic acid, L-glutamine, glycine, L-histidine, L-isoleucine, L-leucine, L-lysine, L-methionine, L-phenylalanine, L-proline, L-serine, L-taurine, L-threonine, L-tryptophan, L-tyrosine, L-valine, and combinations thereof.

The free amino acids may be provided in the form of any pharmaceutically acceptable salt, a non-limiting example of which includes camitine fumarate.

The medical food tablets preferably comprise, in addition to or in place of the free amino acids, typically in addition to, a modified protein hydrolysate selectively low in one or more defined amino acids. These modified hydrolysates are especially useful in providing protein equivalents that contain reduced or low amounts of the offending amino acids associated with any particular metabolic disorder at issue. For example, medical food tablets for use in individuals afflicted with phenylketonuria can be formulated with modified protein hydrolysates that contain reduced or low amounts of phenylalanine to the extent that such reduced or low amounts are safe and effective for use by the particular individual taking the tablets.

The medical food tablets of the present invention therefore contain free amino acids, and are preferably substantially free of intact or partially intact proteins, except as otherwise noted above with respect to modified protein hydrolysates, although it is understood that such proteins can be formulated into the tablets as optional materials in addition to the free amino acid component, where appropriate. In this context, the term “substantially free of intact or partially intact proteins” means that less than about 30%, more preferably less than 10%, even more preferably less than 1%, even more preferably zero percent, of the nitrogen-containing material is in the form of intact or partially intact protein. The medical food tablets may comprise optional flavoring materials (e.g., cocoa powder) that may contain low levels of intact protein, provided that it does not exceed the maximum acceptable level of the offending amino acid.

Non-limiting examples of optional proteins for use in the medical food tablets include those derived from sources such as milk (e.g., casein, whey), animal (e.g., meat, fish), cereal (e.g., rice, corn), vegetable (e.g., soy), or combinations thereof.

2. Sweetening Agent

The medical food tablets of the present invention preferably comprise a sweetening agent suitable for use in oral dosage forms. The sweetening agent can be any material known or otherwise effective as a sweetening material, either alone or in combination with other sweetening agents, in medical food products. The type and amount of sweetening agent is selected so as to provide effective flavor enhancement, modification or masking of taste of the oral medical food tablets.

The sweetening agent is preferably substantially free of reducing sugars. In this context, the term “substantially free of reducing sugars” means that the sweetening agent preferably contains less than about 10%, more preferably less than about 3%, even more preferably zero percent, of a reducing sugar by weight of the total sweetening agent content. Non-limiting examples of reducing sugars to which this preferred limitation applies include fructose, glucose, levulose, dextrose, galactose, lactose, maltose, and other reducing monosaccharides or polysaccharides.

The sweetening agents for use in the compositions include nutritive sweeteners, non-nutritive sweeteners, or combinations thereof. Non-limiting examples of sweetening agents include sucrose, aspartame (not for use in phenylketonuric embodiments) acesulfame K, sucralose, and combinations thereof. The concentration of sweetening agents preferably represents less than 50%, more preferably less than 30%, more preferably from about 5% to about 30%, by weight of the medical food tablet, although it is understood that the tablets may contain higher or lower concentrations depending upon factors such as the type of sweetening agents selected as well as the type and amount of other ingredients used in the tablet formulation.

Some sweetening agents such as sucrose or similar other materials are also useful as flow agents in the formulation process described herein. Although the flow agent in the composition can be a non-sweetening material (e.g., non-sweetening starch, microcystalline cellulose) used in addition to the required sweetening agents, it is preferred so as to minimize tablet size to select at least one sweetening agent that can also act as a flow agent, and thus eliminate the need for excessive amounts of non-sweetening or inert flow agents that would otherwise increase the resulting tablet size.

The sweetening agent preferably includes a combination of sucralose and acesulfame K at a weight ratio of at least about 5.5, preferably from about 5.5 to about 8.0, more preferably from about 5.8 to about 7.4. Moreover, the weight ratio of free amino acids to the combination of sucralose and acesulfame K is preferably at least about 180, more preferably from about 180 to about 400, even more preferably from about 195 to about 379. It has been found that these selected sweetening agents with the defined weight ratios provide an improved flavor profile when used in the context of a free amino acid tablet formulation. Although this particular combination of sweetening agents is most typically used in liquid beverage formulas, it is used at much lower ratios of sucralose to acesulfame K, and it is also not typically used in dry formulations or tablets such as those described herein.

The medical food tablets may further comprise other sweetening or non-sweetening carbohydrate materials in addition to the above-described sweetening agents, although the oral tablets preferably contain minimal amounts of non-sweetening carbohydrates so as to maintain the preferred small tablet size and configuration as described herein. In this context, the tablets preferably contain less than 10%, more preferably less than 5%, even more preferably zero percent, of a non-sweetening carbohydrate in addition to the above-described sweetening agent component.

3. Optional Lipids

The medical food tablets may further comprise a lipid component, but preferably contain less than about 10%, more preferably less than about 5%, even more preferably less than about 1%, of a lipid by weight of the tablet. The tablets preferably contain minimal lipid materials so as to better maintain the small tablet size and configuration as described herein.

When used in the medical food tablets, however, suitable lipids include any lipid material which is known or otherwise suitable for use in oral products, non-limiting examples of which include coconut oil, soy oil, corn oil, olive oil, safflower oil, high oleic safflower oil, sunflower oil, high oleic sunflower oil, palm and palm kernel oils, palm olein, canola oil, marine oils, cottonseed oils, and combinations thereof. Accordingly, the medical food tablets of the present invention are preferably substantially free of any one, some, or all of the above-described lipid materials.

The medical food tablets of the present invention may further comprise, however, lipids in the form of processing lubricants such as ascorbyl palmitate, magnesium stearate, or similar other lubricants, concentrations of which typically range from about 0.25% to about 1%, by weight of the tablet. In addition to providing tablet release during manufacturing processes, the lubricants can also form a thin film around the tablet that further reduces undesirable aromas and/or flavors that might otherwise be associated with the medical food tablet.

The medical food tablets preferably do not contain excessive concentrations of unsaturated fatty acids in the above-described lipid concentration ranges. It has been found that these fatty acids are often susceptible to oxidation when formulated in the medical food tablets, especially when the tablets also contain materials such as iron, copper or other materials that can act as catalysts in the oxidation of unsaturated fatty acids. These lipids are available as free fats or oils or are combined with and adsorbed to a starch matrix.

4. Vitamins and Minerals

The medical food tablets of the present invention preferably further comprise vitamins, minerals, or combinations thereof, suitable for use in the particular medical food prescribed. Any vitamin, mineral or combination thereof that is known or otherwise suitable for use in medical foods can be used in the oral tablets hereof. Non-limiting examples of suitable vitamins include vitamin A as vitamin A palmitate, vitamin A acetate, beta carotene, or combinations thereof; vitamin D; vitamin E; vitamin K; thiamine; vitamin B₁₂; niacin, folic acid; pantothenic acid; biotin; riboflavin; pyridoxine; vitamin C; choline; inositol; salts and derivatives thereof; and combinations thereof. Non-limiting examples of suitable minerals include calcium, chloride, copper, chromium, iodine, iron, magnesium, manganese, molybdenum, phosphorus, potassium, selenium, sodium, zinc, and combinations thereof.

Each of the medical food tablets preferably comprises vitamins and minerals that represent from about 0.1% to about 10%, more typically from about 0.1% to about 8%, even more typically from about 1% to about 5%, of the daily recommended dietary allowance of the individual nutrient materials.

### Optional Ingredients

The medical food tablets of the present invention may further comprise any known or otherwise suitable material for use in medical foods, provided that such optional material is safe and effective for the intended use, is compatible with the other selected ingredients, and does not otherwise unduly impair product performance. Such optional materials will most typically comprise from about 1% to about 30%, more typically from about 5% to about 25%, by weight of the medical food tablet.

Non-limiting examples of such optional materials include various excipients or other materials such as colorants, aroma or flavor masking or enhancing agents, flavorants, aromas, additional nutrients, pharmaceuticals, binders, lubricants, emulsifying agents, disintegrants, flow agents, coating materials, and so forth.

Non-limiting examples of optional excipients include up to about 10% by weight of starch; up to about 15%, typically from about 5% to about 12%, by weight of microcrystalline cellulose, maltodextrin 10-20DE, or combinations thereof; up to about 1.0% by weight of a lubricant such as magnesium stearate and/or ascorbyl palmitate; up to about 1.0% by weight of a glidant such as colloidal silicon dioxide; and up to about 5% by weight of fructooligosaccharide (e.g. inulin), up to about 5% by weight of a binder such as stearic acid.

Sucrose can be used herein as both a sweetening agent and a flow agent, and as a flow agent, some or all can be replaced with carbohydrate excipients such as microcrystalline cellulose, maltodextrin 10-20 DE, starch, fructooligosaccharide-containing materials such as inulin, and combinations thereof, and similar other flow agents, provided that the composition still contains a sweetening agent in addition to or in place of the sucrose.

### Method of Use

The present invention is also directed to a method of providing nutrition to individuals afflicted with any of a number of inherited metabolic disorders responsive to certain protein or amino-acid restricted diets. The method comprises the oral administration, typically on a daily basis, of the medical food tablets of the present invention to provide some or all of the amino acid needs of the individual.

The methods of the present invention are especially useful in providing nutrition for individuals afflicted with metabolic disorders such as phenylketonuria, branched chain keto aciduria, tyrosinemia (Types I-III), isovaleric academia and other disorders of leucine catabolism, homocystinuria, urea cycle disorders, propionic acidemia, methylmalonic acidemia, or glutaric aciduria type 1. These individuals are more likely to comply with their prescribed diet or nutrient restrictions when their medical foods, such as those described herein, are more easily and conveniently transported and consumed throughout the day, and more importantly, have reduced odor and off-flavor as compared to other medical foods containing free amino acids.

A brief summary of some of the metabolic disorders to which the methods and tablets of the present invention can be useful is described in U.S. Pat. Nos. 5,326,569 and 5,587,399 (Acosta, et al.), which descriptions are incorporated herein by reference.

The present invention is especially useful when applied to individuals with phenylketonuria. This disorder results from one's inability to convert excess dietary phenylalanine to tyrosine. As a result of this metabolic block, phenylalanine accumulates in the blood and cerebrospinal fluid some of which is excreted in excess in the urine. Abnormally high levels of phenylalanine are diverted to the formation of orthohydroxyphenylacetic and phenylpyruvic acid, and the corresponding metabolic derivatives, phenylacetic and phenyllactic acids. There is excretion in the urine of these acids. There is interference with the normal metabolism of tyrosine and tryptophan, and unusual intermediary products of these two amino acids appear in the urine.

The present invention is therefore directed to a method of providing nutrition to individuals afflicted with phenylketonuria, wherein the method comprises the administration of the medical food tablets of the present invention that are substantially free of phenylalanine. In this context, the term “substantially free of phenylalanine” means that the tablets are safe for use in individuals afflicted with phenylketonuria, in the prescribed amounts, and will typically contain less than about 5 mg of phenylalanine per 10 grams of protein equivalent consumed from the tablets. The various executions or embodiments of the present invention can provide medically prescribed nutrition to limit the intake of phenylalanine, in order to avoid any excess accumulation of this amino acid in afflicted individuals. A certain minimum amount of essential phenylalanine, however, must still be provided in the diet in order to facilitate normal growth and tissue repair and to maintain the plasma phenylalanine level in the normal range, but it is preferred that such minimum amounts be obtained from ordinary dietary sources other than the medical food tablets of the present invention.

The dosage requirements for each person, including daily total protein and phenylalanine requirements, should be determined by a clinician involved in that person's care. The dosage is dependent upon factors such as age, gene mutation, body weight, and medical condition of the person. Each dose is adjusted to support normal growth, development, and plasma phenylalanine concentrations. The daily phenylalanine allowance is determined relative to the precisely measured protein intake from all nutrition sources.

### Method of Making

The medical food tablets of the present invention are prepared by any known or otherwise effective method for making oral dosage form tablets. Such methods typically involve conventional process steps in which active and excipient ingredients are combined and then ultimately compressed or molded, most typically and preferably compressed, to form the desired tablet configuration.

It has been found that the medical food tablets of the present invention have reduced off-flavors and/or odors when prepared by conventional dry mixing methods. In this context, “dry mixing” refers to the blending of the various dry particulate ingredients without the addition of water or other liquid solvents. The dry mixed ingredients are then compressed into medical food tablets.

It has also been found that the dry mixing process becomes even more effective in reducing off-flavors and odors when any non-flowing particulates in the selected formula are subjected to a conventional dry compaction step prior to the dry mixing step. Dry compaction typically involves either dry granulation (e.g., roller compaction) or slugging (e.g., compressing a material into tablets and then grinding it back down to form large, flowing particles). The dry compacted materials are then dry mixed with the other flowable ingredients, and then compressed into medical food tablets. For purposes of defining these preferred embodiments of the present invention, the non-flowing particulates include one or more, preferably all, of the free amino acids in the formulation, and also preferably choline and any other non-flowing particulate in the formulation.

Roller compaction as described above generally involves a screening procedure that can lead to a narrower particle size distribution with fewer particles at either extreme of the size range. Roller compaction is often better suited for large scale or continuous operations, although slugging and roller compaction are generally considered as equivalent. Suitable roller compaction methods include the use of a “Chilsonator” roller compactor, a description of which is set forth in “The Theory and Practice of Industrial Pharmacy” Lachan et al. 3rd Edn. Lea & Febiger (1986) page 318-320.

The medical food tablets are preferably prepared in an environment having a relative humidity of less than about 30%, more preferably less than about 20%.

The medical food tablets can also be prepared by the methods described herein, wherein the free amino acids are granulated by wet granulation methods and then dried and combined with the other dry particulate ingredients in the tablet formulation. Wet granulation can be used in place of the dry granulation step described herein, to transform a free amino acid powder (or any powder with poor flow properties) with poor flow properties into agglomerated particulates. An example of wet granulation suitable for use herein includes fluid bed granulation involving an aqueous solution containing maltodextrin, sucrose, and an edible gum such as gum acacia, gum arabic, gum tragacanth, and combinations thereof.

The medical food tablets may be film coated by any known or otherwise effective method for film coating such dosage forms. For example, coating polymers can be applied by an aqueous film coating step to avoid the need for organic coating solvents. Non-limiting examples of such film coating polymers include hydroxypropylcellulose, hydroxypropylmethyl cellulose, ethylcellulose (e.g., ethylcellulose in a latex composition as supplied by the FMC Corporation as AQUA-COAT™), methylhydroxyethylcellulose, polyvinylpyrrolidone (POVIDONE™) sodium carboxymethylcellulose and acrylate polymers. Preferred coating polymers include hydroxypropylmethylcellulosealone (HPMC), polyethylene glycol (PEG), and combinations thereof.

The preferred coating polymers can be applied by aqueous film coating techniques and include one or more HPMC's combined with one or more PEG's. The weight ratio of HPMC to PEG in the film coating preferably ranges from about 7.5:1 to about 5.5:1. The HPMC can be applied in the form of a mixture of HPMC 6 cps and 15 cps (centipoises) in a weight ratio ranging from about 2:1 to about 4:1. The PEG can be applied in the form of a mixture of PEG 4000 and 6000 in a weight ratio of from about 1:2 to about 2:1 (PEG can be supplied as PEG 3350 and PEG 4000 can be supplied as PEG 6000). The film coating may further comprise an opacifier (e.g., titanium dioxide) that is preferably present in around a 1:1.+−0.10% proportion with the HPMC in the film coat.

The optional film coating on the medical food tablets is preferably applied by an aqueous film coating process. The solids loading for the aqueous film coat preferably ranges from about 10% to about 30%, typically from about 10% to about 20%, on a weight/volume basis with respect to the coating material.

The optional film coating on the medical food tablets typically represents from about 2% to about 10%, more typically from about 2% to about 5%, by weight of the core tablet prior to coating. The film coating is preferably free of protein, and may further comprise other optional coating ingredients such as FD&C food color lakes.

It has been found that the preferred medical food tablets of the present invention have reduced odor and off-flavors when the amino acid blend, or at least that portion of the blend most responsible for such odor and off flavors, is dry blended into the formulation. It has been found that these amino acid solids, which most typically appear as solid crystals within the tablets, have reduced odor and flavor as compared to the more conventional spray dried amino acids and amino acid blends, especially when the crystalline amino acids are subjected to a dry compaction step prior to a dry mixing step, all of which are described herein.

The medical food tablets are preferably prepared by selection of the odor-producing free amino acids in the amino acid blend used in the formulation, dry mixing the odor-producing free amino acids in the blend with the other ingredients, including the remaining free amino acids if any, and then compressing the dry mixed ingredients to form medical food tablets. Preferably, all of the free amino acids in the blend are dry compacted and then dry mixed with the other ingredients prior to the final compression step.

One skilled in the tablet manufacturing arts will appreciate the normal processing variations of the above-described method in order to obtain the desired characteristics of the medical food tablets, including selection of various diluents, binders, lubricants, disintegrators, coloring agents, flavoring agents, or other processing aids, as well as the use of various tablet machines, mixers and other manufacturing equipment.

### EXAMPLES

The following examples further describe and demonstrate specific embodiments within the scope of the present invention. The examples are given solely for the purpose of illustration and are not to be construed as limitations of the present invention, as many variations thereof are possible without departing from the spirit and scope of the invention. All exemplified amounts are concentrations by weight of the total composition, unless otherwise specified.

Each of the exemplified compositions is prepared by dry compaction (e.g., slugging) of the free amino acids and choline, followed by dry mixing of the dry compacted particulates with the other particulates in the formulation. The dry mixed combination is then compressed into small chewable tablets and dispensed in light resistant dosage kits, all as described hereinafter.

### Example 1

Exemplified is a medical food tablet of the present invention. It is used in accordance with the methods of the present invention to provide nutrition to individuals afflicted with phenylketonuria. Each tablet is round, chewable, weighs about 1 gm, and has a major axis of less than about 1.5 cm and a ratio of the major to minor axis of from about 2 to about 10. Each tablet provides a protein equivalent of about 0.4 grams and contains≦0.2 mg of phenylalanine. Individual consumption of the tablets range from 25-50 tablets, from once to four times per day, typically 2-3 times a day, or as otherwise convenient throughout the day to consume the desired protein equivalents. The specific number of tablets will depend upon the particular nutrient needs of the individual.

Approximately 500 tablets are packaged in an opaque multi-dose prescription vial as part of a dosage form kit that also contains a plurality of opaque, resealable, polymer bags. Individuals take the daily number of tablets from the multi-dose vial and transport them throughout the day in the polymer bags for convenient access and handling of the medical food tablets.

The tablet ingredients are listed in Table 1.1; the resulting nutrient profile is described in Table 1.2.

TABLE 1.1
Medical Food Tablet Ingredients (for phenylketonuria)

| Ingredients | Per 100 g | |
|---|---|---|
| Amino acid premix | 50.65 | g |
| Vitamin/mineral premix | 8.00 | g |
| Artificial masking flavor | 1.12 | g |
| Powdered sucralose | 0.150 | g |
| Acesulfame K | 0.0204 | g |
| m-Inositol | 0.148 | g |
| Potassium citrate | 1.02 | g |
| Sodium citrate | 0.672 | g |
| Choline bitartrate | 0.439 | g |
| Sodium chloride | 1.04 | g |
| Vanilla flavor | 1.40 | g |
| Silicon dioxide | 1.22 | g |
| Sucrose | 20.34 | g |
| Microcrystalline cellulose | 10 | g |
| Cocoa powder | 1.50 | g |
| Chocolate flavor | 1.50 | g |
| Thiamine HCl | 0.0028 | g |
| L-Taurine | 0.186 | g |
| L-Carnitine | 0.084 | g |
| Magnesium stearate* | 0.54 | g |
| Total | 100.00 | |

*Some or all of magnesium stearate can be replaced with ascorbyl palmitate

TABLE 1.2
Medical Food Tablet Nutrient Profile (for phenylketonuria)¹

| Ingredient | Amount | |
|---|---|---|
| Amino acids | gm | |
| L-Alanine | 0.67 | |
| L-Arginine | 0.71 | |
| L-Asparagine | 0.47 | |
| L-Aspartic acid | 0.08 | |
| L-Cystine | 0.10 | |
| L-Glutamic acid | 0.14 | |
| L-Glutamine | 0.84 | |
| Glycine | 0.67 | |
| L-Histidine | 0.28 | |
| L-Isoleucine | 0.72 | |
| L-Leucine | 1.12 | |
| L-Lysine | 0.67 | |
| L-Methionine | 0.20 | |
| L-Proline | 0.96 | |
| L-Serine | 0.51 | |
| L-Threonine | 0.47 | |
| L-Tryptophan | 0.12 | |
| L-Tyrosine | 1.00 | |
| L-Valine | 0.81 | |
| L-Carnitine | 0.013 | |
| L-Taurine | 0.017 | |
| Minerals | | |
| Calcium | 293 | mg |
| Phosphorus | 253 | mg |
| Magnesium | 75 | mg |
| Sodium, | 200 | mg |
| Potassium, | 300 | mg |
| Chloride, | 285 | mg |
| Iodide | 33 | μg |
| Iron | 4.3 | mg |
| Manganese, | 0.26 | mg |
| Zinc, | 3.68 | mg |
| Copper, | 0.33 | mg |
| Selenium, | 11.66 | μg |
| Chromium, | 9.00 | μg |
| Molybdenum, | 10.00 | μg |
| Vitamins | | |
| A² | 730 | IU |
| D | 100 | IU |
| E | 6.0 | IU |
| K₁ | 20 | μg |
| C | 20 | mg |
| Thiamine | 1.08 | mg |
| Riboflavin | 0.6 | mg |
| Pyridoxine | 0.43 | mg |
| B₁₂ | 1.66 | μg |
| Niacin | 5.33 | mg |
| Pantothenic acid | 2.66 | mg |
| Folic acid | 150 | μg |
| Biotin | 33 | μg |
| Choline | 33 | mg |
| Inositol | 23.3 | mg |

¹Profile for 25 tablets containing a total of 6.7 g carbohydrate, 10 g protein equivalent, 1.6 g nitrogen, 62 kcal energy
²Provided as combination of vitamin A and beta-carotene

The exemplified tablets are used to help provide nutrition specifically tailored to the nutrition needs of a particular 6 year old child with phenylketonuria. This particular child is prescribed a daily diet containing 235 mg phenylalanine, 35 gm protein, and 1700 kcal. For breakfast, the child is given 33 gm of Phenex-2 TM powder (amino acid-modified medical food, available from Ross Products Division, Abbott Laboratories, Columbus, Ohio, USA) mixed with water to make 6 fl oz., 20 gm puffed rice, 42 g of banana, and 84 gm of sugar. For lunch at school, the child is given 25 medical food tablets (exemplified above), 100 gm of very low protein bread slices filled with 14 gm butter and 20 gm apple butter, 39 gm raw sliced carrots, and 84 gm raw tangerine. For dinner, the child is given Phenex-2™ powder (33 gm) mixed with water to make 6 fl. oz., baked potato with no skin (60 gm) with 14 gm margarine, and 64 gm cooked snap beans; 66 gm raw sliced tomato; 85 gm frozen sweetened strawberries; 96 gm fruit ice.

The above-described diet, or one differing only in phenylalanine content, is repeated in other young children with appropriate modifications tailored to each individual, except that the Phenex-2™ powder at breakfast and dinner is replaced with a nutritionally equivalent amount of the exemplified medical food tablets.

The exemplified tablets are also used to provide nutrition for a female college student (20 years old) with phenylketonuria. The young woman is prescribed a daily diet containing 220 mg phenylalanine, 50 gm protein, and 2100 kcal. For breakfast, she takes Phenex-2™ powder (50 gm) mixed with water to make 8 fl oz., 13 gm RICE CHEX™ Cereal (General Mills, Inc., Minneapolis, Minn., USA); 74 gm raw sliced strawberries; 56 gm Rich Whip Liquid, and 60 gm sugar. While at school, the young woman has lunch which consists of 38 medical food tablets (exemplified above), 100 gm cooked very low protein pasta with 2 Tbsp vinegar/oil dressing, 39 gm raw carrot sticks, 194 gm canned purple plums, and 1-12 fl. oz. container of a sugar-sweetened, canned carbonated beverage. For dinner, the she takes 38 medical food tablets, 98 gm of sweet potatoes canned in syrup, 20 gm cooked broccoli with 14 gm margarine, 140 gm raw papaya, 1-12 fl oz sugar-sweetened carbonated beverage.

The above-described prescribed diet is repeated, or one differing primarily in phenylalanine content, in other young adults with phenylketonuria with appropriate modifications tailored to each individual, except that the Phenex-2™ powder at breakfast is replaced with a nutritionally equivalent amount of the exemplified medical food tablets.

### Example 2

Exemplified is a medical food tablet of the present invention. It is used in accordance with the methods of the present invention to provide nutrition to individuals afflicted with branched-chain ketoaciduria. Each tablet is round, chewable, weighs about 1 gm, and has a major axis of less than about 1.5 cm and a ratio of the major to minor axis of from about 2 to about 10. Each tablet provides a protein equivalent of about 0.4 grams. Individual consumption of the tablets range from 25-50 tablets, from once to four times per day, typically 2-3 times a day, or as otherwise convenient throughout the day to consume the desired protein equivalents. The specific number of targeted tablets will depend upon the particular nutrient needs of the individual. The tablets are dispensed in light resistant packages as part of a dosage kit as described herein. A daily supply of the tablets are transported to work or school each day in a flexible, resealable pouch, wherein the pouch is part of the dosage kit as described herein.

The tablet ingredients are listed in Table 2.1; the resulting nutrient profile is described in Table 2.2.

TABLE 2.1
Medical Food Tablet Ingredients (for branched-chain ketoaciduria)

| Ingredients | gm/100 gm |
|---|---|
| L-Alanine | 7.651 |
| L-Arginine | 4.229 |
| L-Asparagine monhydrate | 2.455** |
| L-Aspartic acid | 0.389 |
| L-Carnitine | 0.237 |
| L-Cystine dihydrochloride | 0.587** |
| L-Glutamic acid | 0.660 |
| L-Glutamine | 3.691 |
| Glycine | 3.00 |
| L-Taurine | 0.186 |
| L-Histidine | 1.260 |
| L-Lysine acetate | 4.232** |
| L-Methionine | 0.900 |
| L-Phenylalanine | 2.640 |
| L-Proline | 5.400 |
| L-Serine | 2.640 |
| L-Threonine | 2.100 |
| L-Tryptophan | 0.509 |
| L-Tyrosine | 2.669 |
| Vanilla flavor | 1.40 |
| Vit./mineral premix | 8.00 |
| Flavor masking agent | 1.12 |
| Powdered sucralose | 0.150 |
| Acesulfame K | 0.0204 |
| m-Inositol | 0.148 |
| Potassium citrate | 1.02 |
| Sodium citrate | 0.672 |
| Choline bitartrate | 0.439 |
| Silicon dioxide | 1.22 |
| Sucrose | 25.83 |
| Microcrystalline | 10.00 |
| cellulose | |
| Cocoa powder | 1.50 |
| Chocolate flavor | 1.50 |
| Thiamine HCl | 0.0028 |
| Magnesium | 0.54 |
| stearate* | |
| Sodium chloride | 1.04 |

*some or all of magnesium stearate may be replaced with ascorbyl palmitate
**amount expressed as equivalent free amino acid content

TABLET 2.2
Medical Food Tablet Nutrient Profile
(for branched-chain ketoaciduria)¹

| Ingredient | Amount | |
|---|---|---|
| Amino acids | gm | |
| L-Alanine | 1.697 | |
| L-Arginine | 0.940 | |
| L-Asparagine | 0.545** | |
| monhydrate | | |
| L-Aspartic acid | 0.086 | |
| L-Carnitine | 0.53 | |
| L-Cystine | 0.130** | |
| Dihydrochloride | | |
| L-Glutamic acid | 0.147 | |
| L-Glutamine | 0.820 | |
| Glycine | 0.667 | |
| L-Histidine | 0.280 | |
| L-Lysine acetate | 0.940** | |
| L-Methionine | 0.200 | |
| L-Phenylalanine | 0.587 | |
| L-Proline | 1.200 | |
| L-Serine | 0.587 | |
| L-Threonine | 0.467 | |
| L-Tryptophan | 0.120 | |
| L-Tyrosine | 0.593 | |
| Minerals | | |
| Calcium | 293 | mg |
| Phosphorus | 253 | mg |
| Magnesium | 75 | mg |
| Sodium | 200 | mg |
| Potassium | 300 | mg |
| Chloride | 285 | mg |
| Iodide | 33 | μg |
| Iron | 4.3 | mg |
| Manganese | 0.26 | mg |
| Zinc | 3.68 | mg |
| Copper | 0.33 | mg |
| Selenium | 1.66 | μg |
| Chromium | 9.00 | μg |
| Molybdenum | 10.00 | μg |
| Vitamins | | |
| A² | 730 | IU |
| D | 100 | IU |
| E | 6.0 | IU |
| K₁ | 20 | μg |
| C | 20 | mg |
| Thiamine | 1.08 | mg |
| Riboflavin | 0.6 | mg |
| Pyridoxine | 0.43 | mg |
| B₁₂ | 1.66 | μg |
| Niacin | 5.33 | mg |
| Pantothenic acid | 2.66 | mg |
| Folic acid | 150 | μg |
| Biotin | 33 | μg |
| Choline | 33 | mg |
| Inositol | 23.3 | mg |
| L-Taurine | 16.6 | mg |

¹Profile for 25 tablets containing a total of 8 g carbohydrate, 10 g protein equivalent, 1.6 g nitrogen
²Provided as combination of vitamin A and beta-carotene
**Amount expressed as equivalent free amino acid content

What is claimed is:

1. Medical food tablets comprising:
(a) a dry mix blend of free amino acids,
(b) vitamins, minerals, or combinations thereof, and
(c) sweetening agents,
wherein the tablets are prepared by dry mixing the ingredients and compressing the dry mixed ingredients into tablet form.

2. The medical food tablets of claim 1, wherein the free amino acids are dry compacted to form flowable particulates prior to and for use in the subsequent dry mixing step.

3. The medical food tablets of claim 1, wherein the combination of components (a), (b), and (c) represent from about 70% to 100% by weight of the tablet.

4. The medical food tablets of claim 1, wherein each tablet is chewable and has a major axis of less than about 1.5 cm and a major to minor axis ratio of from about 1 to about 10.

5. The medical food tablets of claim 1, wherein each tablet weighs less than 1.5 gm and has a major axis of less than about 1.2 cm.

6. The medical food tablets of claim 1, wherein the sweetening agent comprises a combination of sucralose and acesulfame k at a weight ratio of at least about 5.5.

7. The medical food tablets of claim 1 wherein the blend of free amino acids provides each tablet with a protein equivalent of from about 0.2 grams to about 1 gram, and wherein the free amino acids are selected from the group consisting of L-alanine, L-arginine, L-asparagine, L-aspartic acid, L-cystine, L-glutamic acid, L-glutamine, glycine, L-histidine, L-isoleucine, L-leucine, L-carnitine, L-taurine, L-lysine, L-methionine, L-phenylalanine, L-proline, L-serine, L-taurine, L-threonine, L-tryptophan, L-tyrosine, L-valine, and combinations thereof.

8. The medical food tablets of claim 1, wherein the blend of free amino acids provides each tablet with a protein equivalent of from about 0.2 grams to about 0.6 grams.

9. The medical food tablets of claim 1, wherein the tablets are substantially free of L-phenylalanine.

10. The medical food tablets of claim 1, wherein the tablets collectively contain less than about 5 mg of phenylalanine per each 10 grams of protein equivalent.

11. The medical food tablets of claim 1, wherein the tablets comprise one or more minerals selected from the group consisting of calcium, phosphorus, magnesium, sodium, potassium, chloride, iodide, iron, manganese, zinc, copper, selenium chromium, molybdenum, and combinations thereof.

12. The medical food tablets of claim 1, wherein the tablets comprise one or more vitamins selected from the group consisting of vitamin A, vitamin D, vitamin E, vitamin K1, vitamin C, thiamine, riboflavin, pyridoxine, vitamin B12, niacin, pantothenic acid, folic acid, biotin, choline, inositol, and combinations thereof.

13. The medical food tablets of claim 1, wherein the tablets contain less than 10% by weight of fat, and wherein the sweetening agent contains less than about 10% reducing sugar by weight of the sweetening agent.

14. The medical food tablets of claim 1, wherein the tablets are film coated.

15. The medical food tablets of claim 1, wherein the tablets are chewable.

16. The medical food tablets claim 1, wherein the tablets further comprise a modified protein hydrolysate selectively low in one or more amino acids.

17. The medical food tablets of claim 16, wherein the modified protein hydrolysate is selectively low in phenylalanine, and wherein the tablets collectively contain less than about 5 mg of phenylalanine per each 10 grams of protein equivalent.

18. Chewable medical food tablets comprising:
(a) a blend of free amino acids,
(b) vitamins, minerals, or combinations thereof, and
(c) sweetening agents,
wherein the tablets are chewable and have a major axis of less than about 1.5 cm and a ratio of a major to minor axis from about 1 to about 10.

19. The chewable medical food tablets of claim 18 wherein each tablet weighs from about 0.5 gm to about 1.3 gm and has a major axis of less than about 1.2 cm.

20. The chewable medical food tablets of claim 18 wherein components (a), (b), and (c) represent from about 70% to 100% by weight of the tablets.

21. The chewable medical food tablets of claim 18, wherein the blend of free amino acids provides each tablet with a protein equivalent of from about 0.2 grams to about 1 gram, and wherein the free amino acids are selected from the group consisting of L-alanine, L-arginine, L-asparagine, L-aspartic acid, L-taurine, L-carnitine, L-cystine, L-glutamic acid, L-glutamine, glycine, L-histidine, L-isoleucine, L-leucine, L-lysine, L-methionine, L-phenylalanine, L-proline, L-serine, L-taurine, L-threonine, L-tryptophan, L-tyrosine, L-valine, and combinations thereof.

22. The chewable medical food tablets of claim 18, wherein the blend of free amino acids provides each tablet with a protein equivalent of from about 0.2 grams to about 0.6 grams.

23. The chewable medical food tablets of claim 18, wherein tablets are substantially free of L-phenylalanine.

24. The chewable medical food tablets of claim 18, wherein the tablets collectively contain provide less than about 5 mg of phenylalanine per each 10 grams of protein equivalent.

25. The chewable medical food tablets of claim 18, wherein the tablets comprise one or more minerals selected from the group consisting of calcium, phosphorus, magnesium, sodium, potassium, chloride, iodide, iron, manganese, zinc, copper, selenium chromium, molybdenum, and combinations thereof.

26. The chewable medical food tablets of claim 18, wherein the tablets comprise vitamins selected from the group consisting of vitamin A, vitamin D, vitamin E, vitamin K1, vitamin C, thiamine, riboflavin, pyridoxine, vitamin B12, niacin, pantothenic acid, folic acid, biotin, choline, inositol, and combinations thereof.

27. The chewable medical food tablets of claim 18 wherein the tablets contain less than 10% by weight of fat, and wherein the sweetening agent contains less than about 10% reducing sugar by weight of the sweetening agent.

28. The chewable medical food tablets of claim 18 wherein the tablets are film coated.

29. The chewable medical food tablets of claim 18, wherein the tablets further comprise a modified protein hydrolysate selectively low in one or more amino acids.

30. The chewable medical food tablets of claim 18, wherein the modified protein hydrolysate is selectively low in phenylalanine, and wherein the tablets collectively contain less than about 5 mg of phenylalanine per each 10 grams of protein equivalent.

31. The chewable medical food tablets of claim 18, wherein the sweetening agent comprises a combination of sucralose and acesulfame k at a weight ratio of at least about 5.5.

32. Medical food tablets comprising:
(a) free amino acids,
(b) vitamins, minerals, or combinations thereof,
(c) sweetening agents containing less than 10% by weight of a reducing sugar,
(d) less than about 10% by weight of fat
wherein components (a), (b) and (c) represent at least about 70% by weight of the medical food tablets.

33. The medical food tablets of claim 32, wherein each tablet is chewable and has a major axis of less than about 1.5 cm and a major to minor axis ratio of from about 1 to about 10.

34. The medical food tablets of claim 32, wherein each tablet weighs from about 0.5 gm to about 1.3 gm and has a major axis of less than about 1.2 cm.

35. The medical food tablets of claim 32, wherein the blend of free amino acids provides each tablet with a protein equivalent of from about 0.2 grams to about 1 gram, and wherein the free amino acids are selected from the group consisting of L-alanine, L-arginine, L-asparagine, L-aspartic acid, L-carnitine, L-cystine, L-glutamic acid, L-camitine, L-taurine, L-glutamine, glycine, L-histidine, L-isoleucine, L-leucine, L-lysine, L-methionine, L-phenylalanine, L-proline, L-serine, L-taurine, L-threonine, L-tryptophan, L-tyrosine, L-valine, and combinations thereof.

36. The medical food tablets of claim 32, wherein the blend of free amino acids provides each tablet with a protein equivalent of from about 0.2 grams to about 0.6 grams.

37. The medical food tablets of claim 32, wherein the sweetening agent comprises a combination of sucralose and acesulfame k at a weight ratio of at least about 5.5.

38. The medical food tablets of claim 32, wherein the tablets collectively contain less than about 5 mg of phenylalanine per each 10 grams of protein equivalent.

39. The medical food tablets of claim 32 wherein the tablets comprise one or more minerals selected from the group consisting of calcium, phosphorus, magnesium, sodium, potassium, chloride, iodide, iron, manganese, zinc, copper, selenium chromium, molybdenum, and combinations thereof.

40. The medical food tablets of claim 32, wherein the tablets comprise one or more vitamins selected from the group consisting of vitamin A, vitamin D, vitamin E, vitamin K1, vitamin C, thiamine, riboflavin, pyridoxine, vitamin B12, niacin, pantothenic acid, folic acid, biotin, choline, inositol, combinations thereof.

41. The medical food tablets of claim 32, wherein the tablets are substantially free of unsaturated fat and reducing sugars.

42. The medical food tablets of claim 32, wherein the tablets are film coated and chewable.

43. The medical food tablets of claim 32, wherein the tablets further comprise a modified protein hydrolysate selectively low in one or more amino acids.

44. The medical food tablets of claim 43, wherein the modified protein hydrolysate is selectively low in phenylalanine, and wherein the tablets collectively contain less than about 5 mg of phenylalanine per each 10 grams of protein equivalent.

45. Medical food tablets comprising:
(a) a modified protein hydrolysate selectively low in one or more amino acids,
(b) vitamins, minerals, or combinations thereof, and
(c) a sweetening agent.

46. The medical food tablets of claim 45, wherein the modified protein hydrolysate is selectively low in phenylalanine, and wherein the tablets collectively contain less than about 5 mg of phenylalanine per each 10 grams of protein equivalent.

47. A dosage form kit comprising (a) a first package having a resealable opaque container and a plurality of oral medical food tablets contained therein, and (b) a resealable second package removably positioned in the dosage form kit, said second package used to transport therein at least a portion of the oral medical food tablets away from the dosage form kit, wherein the transported tablets are orally administered within about one week after transport.

48. The dosage form kit of claim 47, wherein the kit comprises medical food tablets comprising:
(a) a dry mix blend of free amino acids,
(b) vitamins, minerals, or combinations thereof, and
(c) sweetening agents,
wherein the tablets are prepared by dry mixing the ingredients and compressing the dry mixed ingredients into tablet form.

49. The dosage form kit of claim 47, wherein the kit comprises medical food tablets comprising:
(a) a blend of free amino acids,
(b) vitamins, minerals, or combinations thereof, and
(c) sweetening agents,
wherein the tablets are chewable and have a major axis of less than about 1.5 cm and a ratio of a major to minor axis from about 1 to about 10.

50. The dosage form kit of claim 47, wherein the kit comprises medical food tablets comprising:
(a) free amino acids,
(b) vitamins, minerals, or combinations thereof,
(c) sweetening agents containing less than 10% by weight of a reducing sugar,
(d) less than about 10% by weight of fat
wherein components (a), (b) and (c) represent at least about 70% by weight of the oral nutritional tablets.

51. A packaged composition comprising a light resistant package and contained therein a plurality of the medical food tablets of claim 1.

52. A packaged composition comprising a light resistant package and contained therein a plurality of the medical food tablets of claim 18.

53. A packaged composition comprising a light resistant package and contained therein a plurality of the medical food tablets of claim 32.

54. A method of providing nutrition for individuals afflicted with phenylketonuria, said method comprising daily oral administration of the medical food tablets of claim 10.

55. A method of providing nutrition for individuals afflicted with phenylketonuria, said method comprising daily oral administration of the medical food tablets of claim 17.

56. A method of providing nutrition for individuals afflicted with phenylketonuria, said method comprising daily oral administration of the medical food tablets of claim 24.

57. A method of providing nutrition for individuals afflicted with phenylketonuria, said method comprising daily oral administration of the medical food tablets of claim 38.

58. A method of making medical food tablets, said method comprising the steps of:
(a) compacting a blend of free amino acid particulates to form a flowable powder,
(b) dry mixing the compacted free amino acid powder with other particulate ingredients, and
(c) compressing the dry mixed ingredients to form the medical food tablets.

59. Themethod of claim 58, wherein the other ingredients comprise a sweetening agent.

60. The method of claim 58, wherein the other ingredients comprise a sweetening agent, vitamins, and minerals.

61. The method of claim 58, wherein the combination free amino acids, sweetening agent, vitamins and minerals represent from about 70% to 100% by weight of the tablet.

62. The method of claim 58, wherein each tablet is chewable and has a major axis of less than about 1.5 cm and a major to minor axis ratio of from about 1 to about 10.

63. The method of claim 58, wherein the blend of free amino acids provides each tablet with a protein equivalent of from about 0.2 grams to about 0.6 grams.

64. The method of claim 58, wherein the tablets collectively contain less than about 5 mg of phenylalanine per each 10 grams of protein equivalent.

65. The method of claim 58, wherein the medical food tablets are film coated, chewable, medical food tablets.
